(19) United States
(12) Patent Application Publication
(10) Pub. No.: US 2013/0336972 A1
(43) Pub. Date: Dec. 19, 2013

(54) RECOMBINANT FC-FUSION PROTEIN OF THE FIFTH FIBRONECTIN TYPE III DOMAIN OF DCC

(71) Applicants: Christian Klein, Bonstetten (CH); Erhard Kopetzki, Penzberg (DE); Gerhard Niederfellner, Oberhausen (DE); Agnes Bernet, Genas (FR); Celine Delloye-Bourgeois, Lyon (FR); Patrick Mehlen, Genas (FR)

(72) Inventors: Christian Klein, Bonstetten (CH); Erhard Kopetzki, Penzberg (DE); Gerhard Niederfellner, Oberhausen (DE); Agnes Bernet, Genas (FR); Celine Delloye-Bourgeois, Lyon (FR); Patrick Mehlen, Genas (FR)

(21) Appl. No.: 13/818,465

(22) PCT Filed: Aug. 26, 2011
(86) PCT No.: PCT/EP11/64733

§ 371 Date: Sep. 3, 2013

(30) Foreign Application Priority Data
Aug. 26, 2010 (EP) 10290459.6

(51) Int. Cl. C07K 16/18

(52) U.S. Cl. CPC C07K 16/18
USPC 424/134.1; 530/387.3; 536/23.4; 435/320.1; 435/328; 435/69.6; 514/44 R; 424/93.21

(57) ABSTRACT

The present invention relates to DCC-fusion proteins, nucleic acid molecules encoding the DCC-fusion proteins, as well as methods for their production and their use in treatment of cancer such as colorectal cancer, NSCLC and metastatic breast cancer. The present invention also relates to methods of treating cancer such as colorectal cancer, NSCLC and metastatic breast cancer by administering DCC-fusion proteins.

### RELATED APPLICATIONS

The present patent application claims priority from EP10290459.6 filed on Aug. 26, 2010 and PCT/EP2011/064733 filed on Aug. 26, 2011.

### BACKGROUND OF THE INVENTION

The present invention relates to a DCC-fusion protein comprising the fifth fibronectin domain (5-fibronectin domain) of Deleted in Colorectal Cancer (DCC) and an antibody Fc part, nucleic acid molecules encoding the same and its production and use for the treatment of cancer.

Netrin-1 is a member of the netrin family and displays an axon navigation cue, both, in an attractive and repulsive context and plays a major role in the development of the nervous system (Serafini, 1996, Cell 87: 1001-1014). The main receptors for netrin-1 are DCC (Deleted in Colorectal Cancer) and UNC5H (UNC5H1, UNC5H2, UNC5H3), which all belong to the so-called dependence receptor family (Keino-Masu, 1996, Cell 87: 175-185; Ackermann, 1997, Nature 386: 838-842; Hong, 1999, Cell 97: 927-941; Mehlen, 1998, Nature 395: 801-804). Dependence receptors share the ability to induce apoptosis in the absence of their respective ligands, whereby this ability is blocked upon binding of the respective ligand (Mehlen, 2004, Cell Mol Life Sci 61: 1854-1866; Bredesen, 2005, Cell Death Differ 12: 1031-1043).

In various human cancers, reduction or loss of expression of DCC and, thus, reduction or loss of DCC-induced apoptosis has been observed (Kinzler, 1996, Proc Natl Acad Sci 100: 4173-4178). Furthermore, it has been observed that also UNC5H genes are downregulated in most colorectal tumours, indicating that the loss of dependence receptor UNC5H represents a selective advantage of tumor cells (Bernet, 2007, Gastroenterology 133: 1840-1848; Shin, 2007, Gastroenterology 133: 1849-1857). However, not only downregulation of the dependence receptors DCC and UNC5H enhances survival of various tumor cells, but also autocrine expression of their ligand netrin-1 has been observed. Particularly, it has been shown that the majority of breast tumors, i.e. metastatic breast cancers, exhibit increased expression of netrin-1 (Fitamant, 2008, Proc Natl Acad Sci 105: 4850-4855). Up to now, it is not yet clear which subdomain of the extracellular part of DCC is responsible for binding of netrin-1. Two subdomains have been discussed in this context, the fifth fibronectin-type III domain (Geisbrecht, 2003, J Biol Chem 278: 32561-32568) and the fourth fibronectin-type III domain (Kruger, 2004, J Neurosci 24: 10826-10834).

As has been shown previously, neutralization of netrin-1 by a DCC-5-fibronectin fusion protein with Glutathione-5-transferase (acting as netrin-1 decoy proteins; also referred to herein as DCC-5Fbn-GST) can induce apoptosis in tumor cells expressing dependence receptors DCC and UNC5H (EP-A1-1989546). A FLAG-tagged DCC-5-fibronectin fusion protein (DCC-5Fbn-GST) can be recombinantly prepared in E. coli and is capable to reduce metastasis of breast cancer cells into the lung over a period of 2 weeks (Fitamant, loc cit). Furthermore, DCC-5Fbn-GST has been demonstrated to increase the cell death percentage of a non-small cell lung cancer (NSCLC) cell line expressing high levels of netrin-1 (Delloye-Bourgeois, 2009, J Natl Cancer Inst 101: 237-247).

However, DCC-5Fbn-GST still bears several weaknesses and must be injected intratumorally in order to be effective. This is probably due to disadvantageous pharmacological properties such as low plasma half-time and fast secretion. Accordingly, there is a need for more effective compounds suitable to treat cancerous diseases associated with reduced or lost dependence receptor-induced apoptosis.

This technical problem has been solved by the embodiments provided herein and the solutions provided in the claims.

### SUMMARY OF THE INVENTION

The present invention relates to a DCC-fusion protein (also named herein Fn5-Fc fusion protein) comprising the fifth fibronectin domain (5-fibronectin domain; Fn5) of Deleted in Colorectal Cancer (DCC) and an antibody Fc-part, particularly the Fc of human IgG1. As has been surprisingly found in the present invention, the C-terminal fusion of an Fc-part of a human IgG1 molecule to the fifth fibronectin-type III domain of DCC leads to an improvement of the pharmacologic properties of the DCC-fusion protein compared to the DCC-fusion proteins of the prior art. In particular, the DCC-fusion protein provided herein exhibits increased affinity to netrin-1 compared to DCC-5Fbn-GST protein.

Furthermore, as will be detailed and exemplified herein, the DCC-fusion protein of the present invention can be produced with high efficiency in HEK 293 cells in transient expressions (>80 mg/l). Additionally, the DCC-fusion protein of the present invention allows for a proper folding of the fifth fibronectin type III-domain of DCC which results in a better binding of the DCC-fusion protein to netrin-1 compared to DCC-5Fbn (the K_{D} of the DCC-fusion protein of the present invention is more than 2-fold lower than for DCC-5Fbn-GST fusion protein, see Keino-Masu, 1996, Cell 87(2):175-85).

### DETAILED DESCRIPTION OF THE FIGURES

The Figures show:

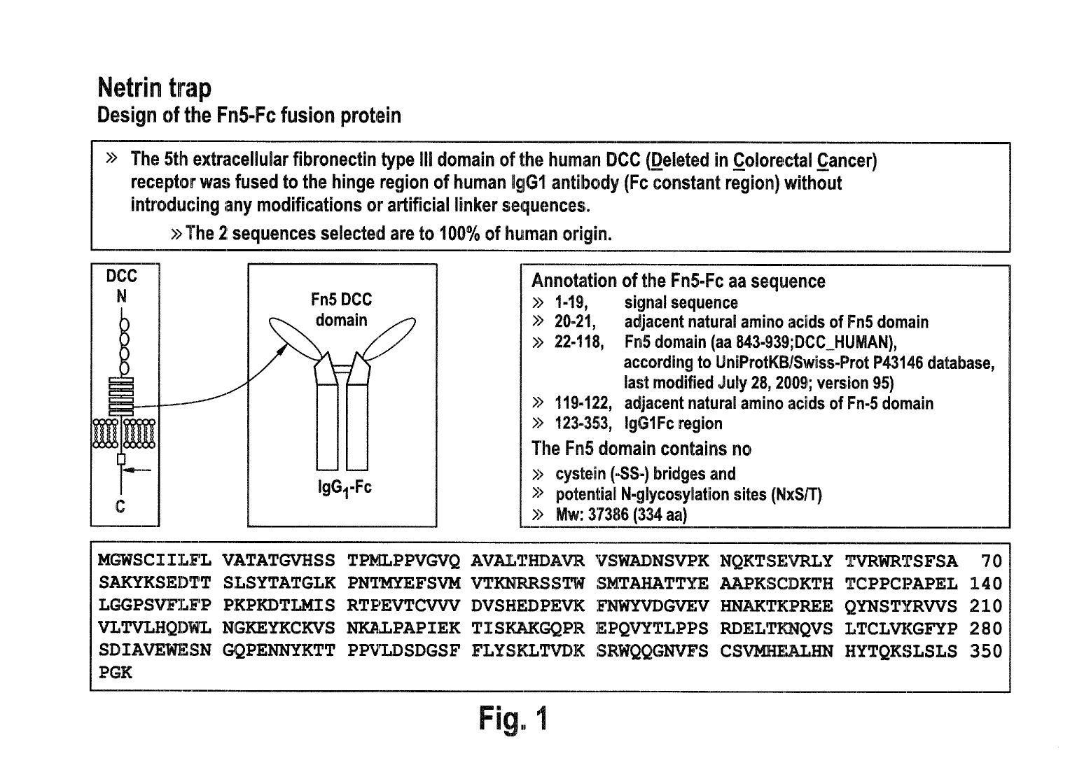

FIG. 1: Schematic presentation of the domain architecture of DCC-fusion protein as provided and described in the present invention.

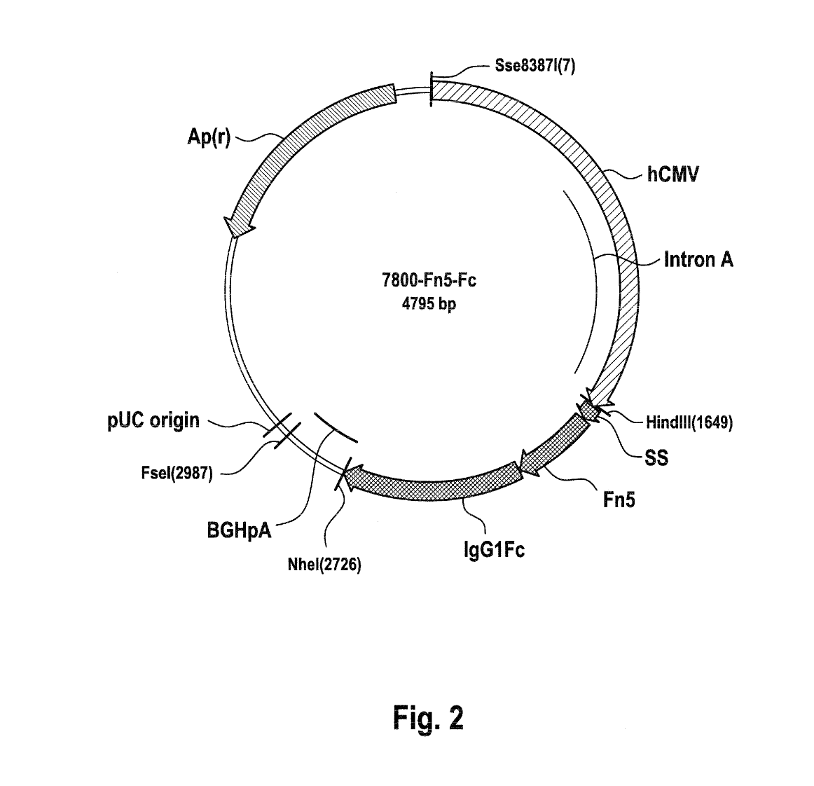

FIG. 2: Plasmid map of DCC-fusion protein (Fn5-Fc; as shown in FIG. 1) expression vector 7800.

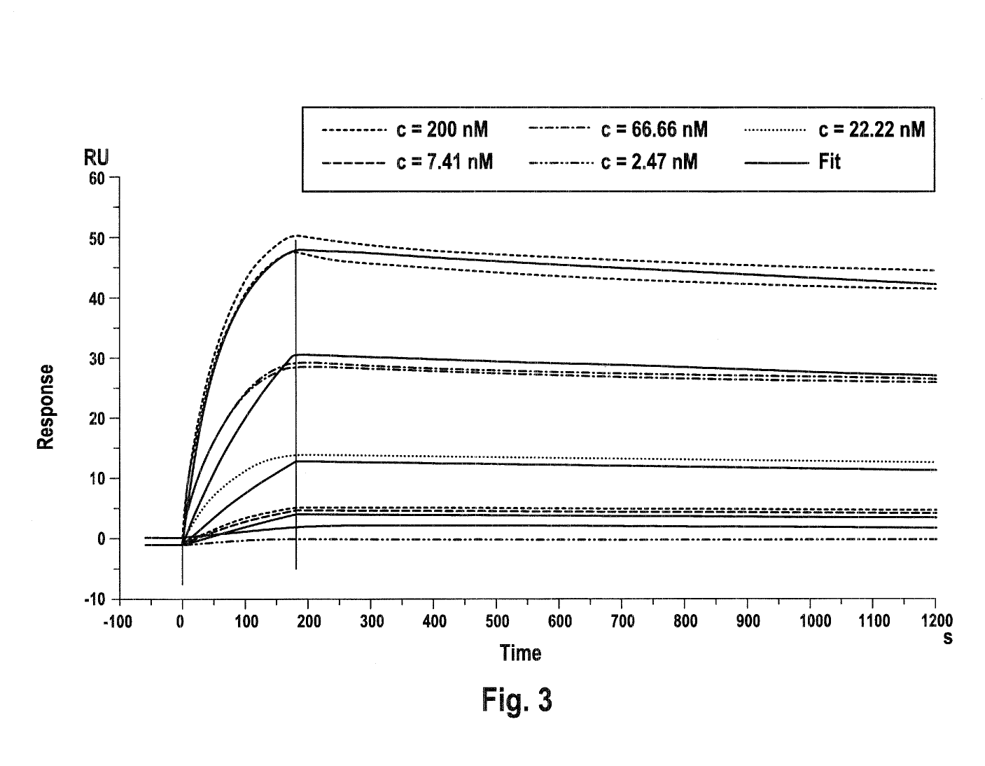

FIG. 3: BIAcore analysis of binding of DCC-fusion protein (SEQ ID NO: 3) as provided and described in the present invention to chicken netrin-1. Fn5 variant 1 was captured on the chip surface via amine coupled capture molecules. A series with increasing concentrations of chicken netrin-1 was injected and the kinetic binding behaviour was monitored by plasmon surface resonance changes. These changes as relative units (R) versus a control chip are recorded on the y-axis over time (x-axis).

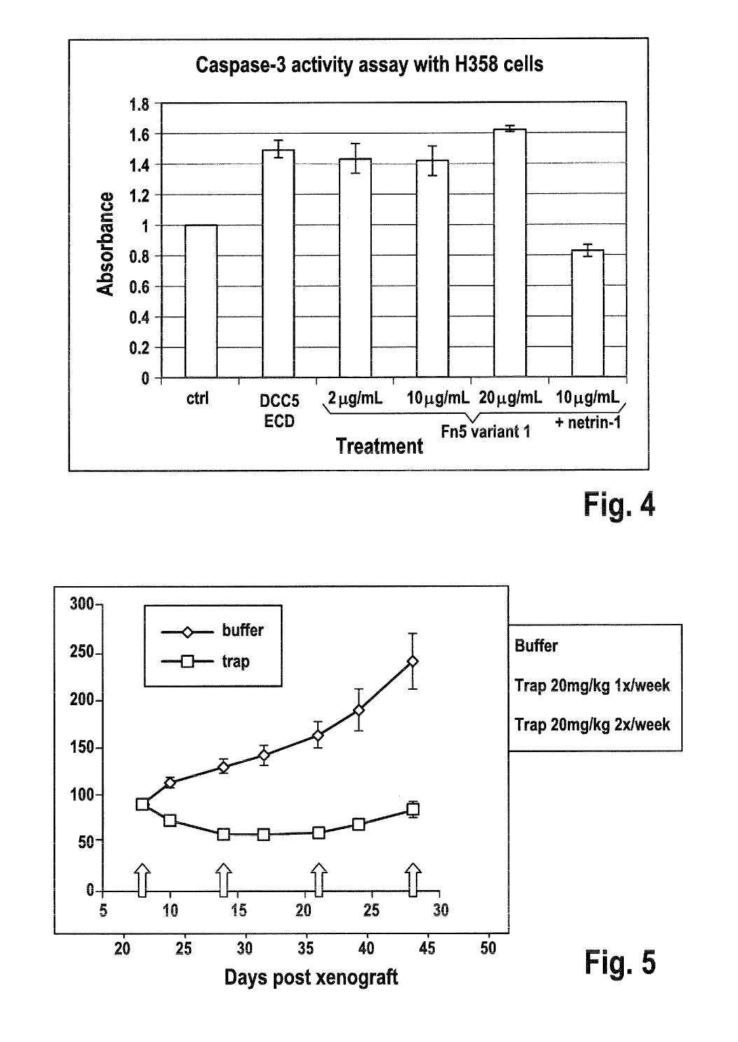

FIG. 4: Caspase-3 activation assay with H-358 cells.
Caspase-3 activity in lysates of differently treated cells is graphed as relative units normalized to buffer-treated control cells (ctrl). As a positive control, cells were treated with an Fc fusion protein comprising the whole extracellular domain of DCC (DCC ECD). The same maximal increase in caspase activity can be induced by the Fn5 variant 1 fusion protein at concentrations of ≧2 μg/ml. Addition of an excess of recombinant netrin-1 blunts caspase-3 activation by 10 μg/ml of Fn5 variant 1.

FIG. 5: In vivo tumor growth inhibition of H358 and A549 xenografts.
Tumor growth of subcutaneous xenografts of H358 (top graph) and A549 (bottom graph) lung cancer cells in nude mice. Tumor volume in mm³ (y-axis) determined by caliper measurements is graphed over time (day 0=day of inoculation) (x-axis). Treatment was started at an average tumor size of ˜100 mm³.

Top graph: Vehicle-treated animals (diamond symbols) show faster H358 tumor cell growth than animals treated once weekly intraperitoneally with 20 mg/kg of the Fn5 variant 1 fusion protein (square symbols). The arrows on the time axis indicate weekly treatments.

Bottom graph: Vehicle-treated animals (diamond symbols) show faster A549 tumor cell growth than animals treated twice per week intraperitoneally with the Fn5 variant 1 fusion protein at 20 mg/kg (triangle symbols). Once weekly treatment at the same dose resulted in an intermediate tumor growth (square symbols). “Trap” means variant 1 fusion protein (SEQ ID NO: 3).

### DETAILED DESCRIPTION OF THE INVENTION

Generally, the DCC-fusion protein provided in the present invention is a binding molecule comprising the fifth fibronectin domain (also referred to as 5-fibronectin domain or Fn5-domain) of DCC and an Fc-part of human IgG1. Particularly, the present invention relates to a DCC-fusion protein comprising or consisting of the amino acid sequence of SEQ ID NO: 2. Amino acids 1 to 19 of SEQ ID NO: 2 show the signal peptide sequence. Amino acids 20 to 353 of SEQ ID NO: 2 as well as SEQ ID NO: 3 show the mature DCC-fusion protein. Accordingly, the present invention relates to a DCC-fusion protein comprising or consisting of the amino acid sequence of SEQ ID NO: 3 (also referred to as Fn5 variant 1). Amino acids 20 and 21 of SEQ ID NO: 2 as well as amino acids 1 and 2 of SEQ ID NO: 3 represent adjacent natural amino acids of the Fn-5 domain. Amino acids 22 to 118 of SEQ ID NO: 2 as well as amino acids 3 to 99 of SEQ ID NO: 3 represent the fifth fibronectin domain (5-fibronectin domain or Fn5-domain) of DCC. Amino acids 119 to 122 of SEQ ID NO: 2 as well as amino acids 100 to 103 of SEQ ID NO: 3 represent adjacent natural amino acids of the Fn-5 domain. Amino acids 123 to 252 of SEQ ID NO: 2 as well as amino acids 104 to 233 of SEQ ID NO: 3 represent the human IgG1 Fc-part.

As described and exemplified herein, the DCC-fusion protein of the present invention has a high binding affinity to netrin-1. Accordingly, the DCC-fusion proteins of the present invention are able to act as decoy molecules binding netrin-1 and, thus, are able to inhibit interaction of netrin-1 and netrin-1 receptors such as DCC and UNC5H (UNC5H1, UNC5H2, UNC5H3). Hence, the present invention relates to DCC-fusion proteins as provided herein for use as a pharmaceutical. Particularly, the present invention relates to DCC-fusion proteins as provided herein for use in treating cancer. Preferably, the cancer to be treated is characterized in that the cancer cells express dependence receptors DCC and/or UNC5H on the surface or show significant upregulation of DCC (Deleted in Colorectal Carcinoma) gene expression (gene ID 1630 (as updated on Aug. 10, 2010) from http://www.ncbi.nlm.nih.gov/gene encoding DCC protein (UniProt ID/version: P43146 (sequence version 2 of May 18, 2010, file version 109 of Aug. 10, 2010))) and/or UNC5H1 (UNC5A) gene expression (gene ID 90249 (as updated on Jun. 26, 2010)), and/or UNC5H2 (UNC5B) gene expression (gene ID 219699 (as updated on Jul. 2, 2010)), and/or UNC5H3 (UNC5C) gene expression (gene ID 8633 (as updated on Aug. 7, 2010)), and/or UNC5H4 (UNC5D) gene expression (gene ID 137970 (as updated on Jul. 2, 2010)) from http://www.ncbi.nlm.nih.gov/gene, encoding UNC-5 homolog proteins (UniProt IDs/versions: Q6ZN44 (entry version 74, sequence version 3), O08722 (entry version 75, sequence version 1), O08747 (entry version 79, sequence version 1), and Q6UXZ4 (entry version 69, sequence version 1). Methods of determining whether a given cell expresses dependence receptors DCC and/or UNC5H on the surface or shows significant upregulation of gene expression are well known in the art and comprise, but are not limited to, IHC (immunohistochemistry) or FACS (Fluorescence activated cell sorting), quantitative PCR (e.g. with hexamer primed cDNA) or alternatively Western Blot paired with chromogenic dye-based protein detection techniques (such as silver or coomassie blue staining) or fluorescence- and luminescence-based detection methods for proteins in solutions and on gels, blots and microarrays, such as immunostaining, as well as immunoprecipitation, ELISA, microarrays, and mass spectrometry. In context of the present invention, examples for cancers to be treated by a DCC-fusion protein of the present invention are lung cancer, non small cell lung cancer (NSCLC), bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers. Specific examples for cancers to be treated by a DCC-fusion protein of the present invention are colorectal cancer, non-small cell lung cancer (NSCLC) and metastatic breast cancer.

Accordingly, the present invention relates to a DCC-fusion protein comprising or consisting of the amino acid sequence of SEQ ID NO: 2 for use in treating cancer. The present invention relates to a DCC-fusion protein comprising or consisting of the amino acid sequence of SEQ ID NO: 3 for use in treating cancer. The present invention relates to a DCC-fusion protein comprising or consisting of the amino acid sequence of SEQ ID NO: 2 for use in treating colorectal cancer. The present invention relates to a DCC-fusion protein comprising or consisting of the amino acid sequence of SEQ ID NO: 2 for use in treating NSCLC. The present invention relates to a DCC-fusion protein comprising or consisting of the amino acid sequence of SEQ ID NO: 2 for use in treating metastatic breast cancer. The present invention relates to a DCC-fusion protein comprising or consisting of the amino acid sequence of SEQ ID NO: 3 for use in treating colorectal cancer. The present invention relates to a DCC-fusion protein comprising or consisting of the amino acid sequence of SEQ ID NO: 3 for use in treating NSCLC. The present invention relates to a DCC-fusion protein comprising or consisting of the amino acid sequence of SEQ ID NO: 3 for use in treating metastatic breast cancer.

The present invention relates to a nucleic acid molecule encoding a DCC-fusion protein described and provided herein. Accordingly, the present invention relates to a nucleic acid molecule encoding the amino acid sequence of SEQ ID NO: 2. The present invention also relates to a nucleic acid molecule encoding the amino acid sequence of SEQ ID NO: 3. Particularly, the present invention relates to a nucleic acid molecule comprising or consisting of the nucleotide sequence of SEQ ID NO: 1. Nucleotides 16 to 1074 of SEQ ID NO: 1 represent the ORF encoding the amino acid sequence of SEQ ID NO: 2. Accordingly, the present invention relates to a nucleic acid molecule comprising or consisting of the nucleotide sequence of nucleotides 16 to 1074 SEQ ID NO: 1. Nucleotides 73 to 1074 of SEQ ID NO: 1 represent the ORF encoding the amino acid sequence of SEQ ID NO: 3. Accordingly, the present invention relates to a nucleic acid molecule comprising or consisting of the nucleotide sequence of nucleotides 73 to 1074 of SEQ ID NO: 1.

The nucleic acid molecule of the present invention may be DNA molecules or RNA molecules. They may also be nucleic acid analogues, such as oligonucleotide thiophosphates, substituted ribo-oligonucleotides, LNA molecules, PNA molecules, GNA (glycol nucleic acid) molecules, TNA (threose nucleic acid) molecules, morpholino polynucleotides, or antagomir (cholesterol-conjugated) nucleic acid molecules or any modification thereof as known in the art (see, e.g., U.S. Pat. No. 5,525,711, U.S. Pat. No. 4,711,955, U.S. Pat. No. 5,792,608 or EP 302175 for examples of modifications). Nucleic acid molecules in context of the present invention may be naturally occurring nucleic acid residues or artificially produced nucleic acid residues. Examples for nucleic acid residues are adenine (A), guanine (G), cytosine (C), thymine (T), uracil (U), xanthine (X), and hypoxanthine (HX). In context of the present invention, thymine (T) and uracil (U) may be used interchangeably depending on the respective type of nucleic acid molecule. For example, as the skilled person is well aware of, a thymine (T) as part of a DNA corresponds to an uracil (U) as part of the corresponding transcribed mRNA. The nucleic acid molecule of the present invention may be single- or double-stranded, linear or circular, natural or synthetic, and, if not indicated otherwise, without any size limitation. The nucleic acid molecule may also comprise a promoter as further detailed herein below. The promoter may be homologous or heterologous. In a particular embodiment, the nucleic acid molecule provided herein is under the control of this promoter.

Generally, as used herein, a polynucleotide comprising the nucleic acid sequence of a sequence provided herein may also be a polynucleotide consisting of said nucleic acid sequence.

Furthermore, in accordance with the present invention, the nucleic acid molecule of the present invention may be cloned into a vector. The term “vector” as used herein particularly refers to plasmids, cosmids, viruses, bacteriophages and other vectors commonly used in genetic engineering. In a preferred embodiment, these vectors are suitable for the to transformation of cells, eukaryotic cells like fungal cells, cells of microorganisms such as yeast or prokaryotic cells. In a particularly preferred embodiment, such vectors are suitable for stable transformation of bacterial cells, for example to transcribe the nucleic acid molecule of the present invention. For example, the vector may be pUC18 or 7800 as shown in FIG. 2 and as described in Example 1 herein. The present invention thus relates to a vector such as pUC18 or 7800 containing a nucleic acid molecule of the present invention. The present invention therefore relates to a vector such as pUC18 or 7800 containing a nucleic acid molecule encoding the amino acid sequence of SEQ ID NO: 2. The present invention also relates to a vector such as pUC118 or 7800 containing a nucleic acid molecule encoding the amino acid sequence of SEQ ID NO: 3. Particularly, the present invention relates to a vector such as pUC18 or 7800 containing a nucleic acid molecule comprising or consisting of the nucleotide sequence of SEQ ID NO: 1. The present invention also relates to a vector such as pUC18 or 7800 containing a nucleic acid molecule comprising or consisting of the nucleotide sequence of nucleotides 16 to 1074 SEQ ID NO: 1. The present invention also relates to a vector such as pUC18 or 7800 containing a nucleic acid molecule comprising or consisting of the nucleotide sequence of nucleotides 73 to 1074 SEQ ID NO: 1. Generally, the vector may be capable of expressing said nucleic acid molecule in a eukaryotic host cell.

Accordingly, in one aspect of the invention, the vector as provided is an expression vector. Generally, expression vectors have been widely described in the literature. As a rule, they may not only contain a selection marker gene and a replication-origin ensuring replication in the host selected, but also a promoter, and in most cases a termination signal for transcription. Between the promoter and the termination signal there is preferably at least one restriction site or a polylinker which enables the insertion of a nucleic acid sequence/molecule desired to be expressed.

It is to be understood that when the vector provided herein is generated by taking advantage of an expression vector known in the prior art that already comprises a promoter suitable to be employed in context of this invention, for example expression of a DCC-fusion protein as described herein, the nucleic acid molecule is inserted into that vector in a manner that the resulting vector comprises preferably only one promoter suitable to be employed in context of this invention. The promoter may generally be heterologous or homologous. The vector described herein may also encompass more than one promoter, each respective promoter may be heterologous or homologous. The skilled person knows how such insertion can be put into practice. For example, the promoter can be excised either from the nucleic acid construct or from the expression vector prior to ligation.

The proteins according to the invention are preferably produced by recombinant means. Preferably, the protein expression is in eukaryotic cells with subsequent isolation of the polypeptide and usually purification to a pharmaceutically acceptable purity. For the protein expression, nucleic acids encoding the protein thereof are inserted into expression vectors by standard methods. Expression is performed in appropriate stable eukaryotic host cells like CHO cells, NS0 cells, SP2/0 cells, HEK293 cells, COS cells, and the protein is recovered from the cells (supernatant or cells after lysis).

In an additional embodiment, the nucleic acid molecule of the present invention and/or the vector into which the polynucleotide described herein is cloned may be transduced, transformed or transfected or otherwise introduced into a host cell. For example, the host cell is a eukaryotic or a prokaryotic cell, preferably a eukaryotic cell. As a non-limiting example, the host cell is a mammalian cell. The host cell described herein is intended to be particularly useful for generating the DCC-fusion protein described and provided in the present invention.

Generally, the host cell described hereinabove may be a prokaryotic or eukaryotic cell, preferably a eukaryotic cell, comprising a nucleic acid molecule provided in the present invention (e.g., comprising or consisting of the sequence of SEQ ID NO: 1, nucleotides 16 to 1074 of SEQ ID NO: 1 or nucleotides 73 to 1074 of SEQ ID NO: 1) or the vector described herein or a cell derived from such a cell and containing the nucleic acid molecule or the vector described herein. In a preferred embodiment, the host cell comprises, i.e. is genetically modified with the nucleic acid molecule of the present invention or the vector described herein in such a way that it contains the nucleic acid molecule of the present invention integrated into the genome. For example, such host cell described herein may be a human, yeast, or fungus cell. In one particular aspect, the host cell is capable to transcribe the nucleic acid molecule of the present invention. An overview of examples of different corresponding expression systems to be used for generating the host cell described herein is for instance contained in Methods in Enzymology 153 (1987), 385-516, in Bitter (Methods in Enzymology 153 (1987), 516-544), in Sawers (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), and in Griffiths (Methods in Molecular Biology 75 (1997), 427-440). The transformation or genetically engineering of the host cell with a nucleic acid molecule of the present invention or vector described herein can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990. In one aspect of the present invention, the host cell comprising the nucleic acid molecule provided herein or a vector described herein may be a HEK293 cell or a HEK293-Freestyle cell (human embryonic kidney cell line 293, Invitrogen). Accordingly, the present invention relates to an HEK293 cell or HEK293-Freestyle cell comprising a nucleic acid molecule encoding the amino acid sequence of SEQ ID NO: 2. The present invention also relates to an HEK293 cell or HEK293-Freestyle cell comprising a nucleic acid molecule encoding the amino acid sequence of SEQ ID NO: 3. The present invention also relates to an HEK293 cell or HEK293-Freestyle cell comprising a nucleic acid molecule comprising or consisting of the nucleotide sequence of SEQ ID NO: 1. The present invention also relates to an HEK293 cell or HEK293-Freestyle cell comprising a nucleic acid molecule comprising or consisting of the nucleotide sequence of nucleotides 16 to 1074 SEQ ID NO: 1. The present invention also relates to an HEK293 cell or HEK293-Freestyle cell comprising a nucleic acid molecule comprising or consisting of the nucleotide sequence of nucleotides 73 to 1074 SEQ ID NO: 1.

The present invention relates to an HEK293 cell or HEK293-Freestyle cell comprising a vector such as pUC18 or 7800 as described and provided herein containing a nucleic acid molecule encoding the amino acid sequence of SEQ ID NO: 2. The present invention also relates to an HEK293 cell or HEK293-Freestyle cell comprising a vector such as pUC18 or 7800 as described and provided herein containing a nucleic acid molecule encoding the amino acid sequence of SEQ ID NO: 3. The present invention also relates to an HEK293 cell or HEK293-Freestyle cell comprising a vector such as pUC18 or 7800 as described and provided herein containing a nucleic acid molecule comprising or consisting of the nucleotide sequence of SEQ ID NO: 1. The present invention also relates to an HEK293 cell or HEK293-Freestyle cell comprising a vector such as pUC18 or 7800 as described and provided herein containing a nucleic acid molecule comprising or consisting of the nucleotide sequence of nucleotides 16 to 1074 SEQ ID NO: 1. The present invention also relates to an HEK293 cell or HEK293-Freestyle cell comprising a vector such as pUC18 or 7800 as described and provided herein containing a nucleic acid molecule comprising or consisting of the nucleotide sequence of nucleotides 73 to 1074 SEQ ID NO: 1.

The present invention relates to a method for producing the DCC-fusion protein as provided and described herein, comprising the steps of expressing a nucleic acid molecule as provided and described herein in a host cell as described herein and recovering the DCC-fusion protein from said cell or the cell culture supernatant. Accordingly, the present invention relates to a method for producing a DCC-fusion protein comprising or consisting of the amino acid sequence of SEQ ID NO: 2, comprising the steps of expressing a nucleic acid molecule encoding the amino acid sequence of SEQ ID NO: 2 in a host cell (e.g., HEK293 cell or HEK293-Freestyle cell) and recovering the DCC-fusion protein from said cell or the cell supernatant. Accordingly, the present invention relates to a method for producing a DCC-fusion protein comprising or consisting of the amino acid sequence of SEQ ID NO: 3, comprising the steps of expressing a nucleic acid molecule encoding the amino acid sequence of SEQ ID NO: 3 in a host cell (e.g., HEK293 cell or HEK293-Freestyle cell) and recovering the DCC-fusion protein from said cell or the cell supernatant. The present invention relates to a DCC-fusion protein obtained or obtainable by the method provided and described herein.

The present invention relates to compositions comprising a DCC-fusion protein as provided herein, a nucleic acid molecule as provided herein, a vector as described herein, and/or a host cell as described herein. The composition comprising a DCC-fusion protein as provided herein, a nucleic acid molecule as provided herein, a vector as described herein, and/or a host cell as described herein may further comprise a pharmaceutically acceptable carrier, excipient and/or diluent. Accordingly, the present invention relates to a DCC-fusion protein as provided herein, a nucleic acid molecule as provided herein, a vector as described herein, and/or a host cell as described herein for use as a pharmaceutical, optionally together with a pharmaceutically acceptable carrier, excipient and/or diluent. Accordingly, the present invention also relates to a pharmaceutical composition comprising a DCC-fusion protein as provided herein, a nucleic acid molecule as provided herein, a vector as described herein, and/or a host cell as described herein and optionally further comprising a pharmaceutically acceptable carrier, excipient and/or diluent. Generally, examples of suitable pharmaceutical carriers are well known in the art and include phosphate buffered saline solutions, water, emulsions, such as oil/water emulsions, various types of wetting agents, sterile solutions etc. Pharmaceutical compositions comprising such carriers can be formulated by well known conventional methods. These pharmaceutical compositions can be administered to a subject at a suitable dose, i.e. at least 1 mg/kg body weight, e.g. about 10 mg/kg body weight to about 100 mg/kg body weight of the subject in which cancer, particularly colorectal cancer, NSCLC or metastatic breast cancer is to be treated. Administration of the composition may be effected or administered by different ways, e.g., enterally, orally (e.g., pill, tablet, buccal, sublingual, disintegrating, capsule, thin film, liquid solution or suspension, powder, solid crystals or liquid), rectally (e.g., suppository, enema), via injection (e.g., intravenously, subcutaneously, intramuscularly, intraperitoneally, intradermally) via inhalation (e.g., intrabronchially), topically, vaginally, epicutaneously, or intranasally. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The compositions and pharmaceutical compositions comprising a DCC-fusion protein as provided herein, a nucleic acid molecule as provided herein, a vector as described herein, and/or a host cell and optionally a pharmaceutically acceptable carrier, excipient and/or diluent as described herein may be administered locally or systemically. Administration will preferably be intravenously or subcutaneously. The compositions and pharmaceutical compositions may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, also doses below or above of the exemplary ranges described hereinabove are envisioned, especially considering the aforementioned factors.

As already mentioned, the compositions described herein comprising a DCC-fusion protein as provided herein, a nucleic acid molecule as provided herein, a vector as described herein, and/or a host cell as described herein may be used to treat cancer, particularly colorectal cancer, NSCLC or metastatic breast cancer in a subject. Accordingly, the present invention relates to pharmaceutical compositions comprising a DCC-fusion protein as provided herein, a nucleic acid molecule as provided herein, a vector as described herein, and/or a host cell as described herein for use in treating cancer, particularly colorectal cancer, NSCLC or metastatic breast cancer. As already mentioned, the pharmaceutical composition may further comprise a pharmaceutically acceptable carrier, excipient and/or diluent as described hereinabove. The present invention therefore relates to a pharmaceutical composition comprising a DCC-fusion protein comprising or consisting of the amino acid sequence of SEQ ID NO: 2 and a pharmaceutically acceptable carrier, excipient and/or diluent as described hereinabove for use in treating cancer, particularly colorectal cancer, NSCLC or metastatic breast cancer. The present invention therefore relates to a pharmaceutical composition comprising a DCC-fusion protein comprising or consisting of the amino acid sequence of SEQ ID NO: 3 and a pharmaceutically acceptable carrier, excipient and/or diluent as described hereinabove for use in treating cancer, particularly colorectal cancer, NSCLC or metastatic breast cancer. The present invention relates to a pharmaceutical composition comprising a nucleic acid molecule encoding the amino acid sequence of SEQ ID NO: 2 and a pharmaceutically acceptable carrier, excipient and/or diluent as described hereinabove for use in treating cancer, particularly colorectal cancer, NSCLC or metastatic breast cancer. The present invention relates to a pharmaceutical composition comprising a nucleic acid molecule encoding the amino acid sequence of SEQ ID NO: 3 and a pharmaceutically acceptable carrier, excipient and/or diluent as described hereinabove for use in treating cancer, particularly colorectal cancer, NSCLC or metastatic breast cancer. The present invention relates to a pharmaceutical composition comprising a nucleic acid molecule comprising or consisting of the nucleotide sequence of SEQ ID NO: 1 and a pharmaceutically acceptable carrier, excipient and/or diluent as described hereinabove for use in treating cancer, particularly colorectal cancer, NSCLC or metastatic breast cancer. The present invention relates to a pharmaceutical composition comprising a nucleic acid molecule comprising or consisting of the nucleotide sequence of nucleotides 16 to 1074 of SEQ ID NO: 1 and a pharmaceutically acceptable carrier, excipient and/or diluent as described hereinabove for use in treating cancer, particularly colorectal cancer, NSCLC or metastatic breast cancer. The present invention relates to a pharmaceutical composition comprising a nucleic acid molecule comprising or consisting of the nucleotide sequence of nucleotides 73 to 1074 of SEQ ID NO: 1 and a pharmaceutically acceptable carrier, excipient and/or diluent as described hereinabove for use in treating cancer, particularly colorectal cancer, NSCLC or metastatic breast cancer. The present invention relates to a pharmaceutical composition comprising a vector as described containing a nucleic acid molecule comprising or consisting of the nucleotide sequence of SEQ ID NO: 1 and a pharmaceutically acceptable carrier, excipient and/or diluent as described hereinabove for use in treating cancer, particularly colorectal cancer, NSCLC or metastatic breast cancer. The present invention relates to a pharmaceutical composition comprising a vector as described herein containing a nucleic acid molecule comprising or consisting of the nucleotide sequence of nucleotides 16 to 1074 of SEQ ID NO: 1 and a pharmaceutically acceptable carrier, excipient and/or diluent as described hereinabove for use in treating cancer, particularly colorectal cancer, NSCLC or metastatic breast cancer. The present invention relates to a pharmaceutical composition comprising a vector as described herein containing a nucleic acid molecule comprising or consisting of the nucleotide sequence of nucleotides 73 to 1074 of SEQ ID NO: 1 and a pharmaceutically acceptable carrier, excipient and/or diluent as described hereinabove for use in treating cancer, particularly colorectal cancer, NSCLC or metastatic breast cancer. The present invention relates to a pharmaceutical composition comprising a host cell as described herein containing a nucleic acid molecule or a vector as provided and described herein and a pharmaceutically acceptable carrier, excipient and/or diluent as described hereinabove for use in treating cancer, particularly colorectal cancer, NSCLC or metastatic breast cancer.

The present invention further relates to the use of a DCC-fusion protein as provided herein, a nucleic acid molecule as provided herein, a vector or a host cell as described herein, for the manufacture of a medicament for treating cancer, particularly colorectal cancer, NSCLC or metastatic breast cancer. The present invention also relates to a method of treating cancer, particularly colorectal cancer, NSCLC or metastatic cancer, in a subject by administering a DCC-fusion protein as provided herein, a nucleic acid molecule as provided herein, a vector or a host cell as described herein to the subject in need thereof.

Generally, the dosages of the compounds and compositions as described and provided herein to be administered to the subject as mentioned above may be chosen for each and every pharmaceutical embodiment and employment as specified and described herein.

Generally, the subject to be treated in context of the present invention may be mammal and is preferably human.

The Examples illustrate the invention but are not limitative thereof.

### Example 1

### Fn5-Fc Fusion Proteins

### Plasmid Construction

Standard methods were used to manipulate DNA as described in Sambrook, J. et al., Molecular cloning: A laboratory manual; Cold Spring Harbor Laboratory Press, Cold Spring Harbor, N.Y., 1989. The molecular biological reagents were used according to the manufacturer's instructions. Desired gene segments were prepared by gene synthesis. The synthesized gene fragments were cloned into a specified expression vector. The DNA sequence of the subcloned gene fragments were confirmed by DNA sequencing.

### Expression Plasmid 7800

Vector 7800 is an expression plasmid e.g. for transient expression of an artificial Ig Fc fusion protein in which the fifth extracellular fibronectin type III domain of the human DCC (Deleted in Colorectal Cancer) receptor is fused to the hinge region of human IgG1 antibody (Fc constant region; Hinge-CH2-CH3) without introducing any modifications or artificial linker sequences; cf. FIG. 2.

A DNA segment of 1084 bps (SEQ ID NO: 1) was prepared by chemical gene synthesis and PCR techniques coding for the open reading frame (ORF) of the desired DCC-fusion protein (Fn5-Fc fusion protein) (SEQ ID NO: 2). The DCC-fusion protein (Fn5-Fc fusion protein) is composed of a murine immunoglobulin heavy chain signal sequence (amino acids 1 to 19 of SEQ ID NO: 2), the fifth extracellular fibronectin type III domain of the human DCC receptor (amino acids 22 to 118 of SEQ ID NO: 2; amino acids 843 to 939 of DCC (Deleted in Colorectal Carcinoma; amino acid sequence: UniProt ID: P43146 (sequence version 2) including adjacent natural amino acids of the fifth fibronectin type III domain (Fn5 domain) at the N-terminal end (amino acids 20 to 21 of SEQ ID NO: 2) and the C-terminal end (amino acids 119 to 122 of SEQ ID NO: 2) of the Fn5 domain, and the human IgG1 antibody Fc constant region (amino acids 123 to 353 of SEQ ID NO: 2). For easy assembly of the Fn5-Fc expression cassette, the chemically prepared DNA segment is flanked by a unique HindIII and NheI restriction endonuclease cleavage site at the 5′- and the 3′-end, respectively. The Fn5-Fc structural gene (ORF; nucleotides 16 to 1074 of SEQ ID NO: 1) was joint to the immediate early enhancer and promoter from the human cytomegalovirus (hCMV) and the bovine growth hormone (bGH) polyadenylation site.

Beside the expression cassette for the DCC-fusion protein (Fn5-Fc fusion protein), the plasmid comprises:
an origin of replication from the vector pUC18 which allows replication of this plasmid in E. coli, and
a β-lactamase gene which confers ampicillin resistance in E. coli.

The transcription unit of the DCC-fusion protein's (Fn5-Fc fusion protein's) encoding sequence (cf. SEQ ID NO: 1) comprises the following elements:
the immediate early enhancer and promoter from the human cytomegalovirus,
a 5′-untranslated region of a human antibody germline gene,
a murine immunoglobulin heavy chain signal sequence,
the Fn5-Fc fusion protein encoding sequence (nucleotides 16 to 1074 of SEQ ID NO: 1), and
the bovine growth hormone (bGH) polyadenylation (“poly A”) signal sequence.

The plasmid map of expression plasmid 7800 is shown in FIG. 2. The amino acid sequence of the mature DCC-fusion protein (i.e. without signal sequence) is shown in SEQ ID NO: 3 (Fn5 variant 1).

Vector 7809 is an expression plasmid for a Fn5-Fc fusion protein variant which differs from DCC-fusion protein (Fn5-Fc fusion protein; SEQ ID NO: 3 for the mature protein) used in the construction of 7800 by a single point mutation within the antibody hinge constant region resulting in a Cys to Ala substitution at amino acid position 107 (compared to mature DCC-fusion protein (Fn5-Fc fusion protein) as shown in SEQ ID NO: 3) as shown in SEQ ID NO: 4.

### Example 2

### Transient Transfection and Expression

Recombinant proteins according to the invention as exemplified in Example 1 were obtained by transient transfection of HEK293-Freestyle cells (human embryonic kidney cell line 293, Invitrogen) growing in suspension. The transfected cells were cultivated in F17 medium (Gibco) or Freestyle 293 medium (Invitrogen), supplemented with 6 mM Glutamine, either Ultra-Glutamine (Biowhittake/Lonza) or L-Glutamine (Sigma), with 8% CO₂ at 37° C. in shake flasks in the scale of 30 ml to 250 ml medium. For transfection Fectin (Invitrogen) was used in a ratio of reagent (μl) to DNA (μg) of 4:3. Polypeptides containing cell culture supernatants were harvested at day 6 to 8 after transfection. General information regarding the recombinant expression of human immunoglobulins in, e.g., HEK293 cells is given in: Meissner, P. et al., Biotechnol. Bioeng. 75 (2001) 197-203. The DCC-fusion protein (SEQ ID NO: 3) could be secreted with high efficiency at a rate of at least 100 mg/L at transient expression in HEK293-Freestyle cells

### Example 3

### Expression Analysis Using SDS-PAGE

LDS sample buffer, fourfold concentrate (4×LDS): 4 g glycerol, 0.682 g TRIS (tris-(hydroxymethyl)-aminomethane), 0.666 g TRIS-HCl (tris-(hydroxymethyl)-aminomethane-hydrochloride), 0.8 g LDS (lithium dodecyl sulfate), 0.006 g EDTA (ethylene diamin tetra acid), 0.75 ml of a 1% by weight (w/w) solution of Serva Blue G250 in water, 0.75 ml of a 1% by weight (w/w) solution of phenol red, add water to make a total volume of 10 ml.

The culture broths containing the secreted protein were centrifuged to remove cells and cell debris. An aliquot of the clarified supernatants were admixed with ¼ volumes (v/v) of 4×LDS sample buffer and 1/10 volume (v/v) of 0.5 M 1,4-dithiotreitol (DTT). Then the samples were incubated for 10 min. at 75° C. and protein separated by SDS-PAGE. The NuPAGE® Pre-Cast gel system (Invitrogen) was used according to the manufacturer's instruction. In particular, 10% NuPAGE® Novex® Bis-TRIS Pre-Cast gels (pH 6.4) and a NuPAGE® MES running buffer was used. The mature DCC-fusion proteins (Fn5 variant 1: SEQ ID NO: 3; and mutated Fn5 variant 1: SEQ ID NO: 4) could be clearly detected after staining with Coomassie Brilliant Dye. The expression yield in the culture supernatant was >100 mg/L. In comparison, expression yields of other constructs comprising the Fn5 variant 2 (SEQ ID NO: 6) or the Fn4+Fn5 variants 1 and 2 (SEQ ID NO: 5 and SEQ ID NO: 7, respectively) (which were expressed analogously as described in Example 1 hereinabove based on the plasmids 7801, 7802 and 7803) showed only low expression yields. Results are shown in Table 1.

TABLE 1
Results of expression and analytics

| | | | | Expression | Western | |
|---|---|---|---|---|---|---|
| | | | | yield μg/ml | Blot | Western |
| Plasmid | | | MG | (supernatant | (super- | Blot |
| Nr. | Characteristic | Sequence | kDa | day 6) | natant | (Cells) |
| 7800 | Fn5 variant 1 | SEQ ID | 37.5 | 118.2-125 | ok | ok |
| | | NO: 3 | | | | |
| 7809 | mutated Fn5 variant | SEQ ID | 37.5 | 134.5 | ok | ok |
| | 1; Cys to Ala | NO: 4 | | | | |
| | mutation at amino | | | | | |
| | acid position 107 | | | | | |
| | (C107A mutant) | | | | | |
| 7802 | Fn5 variant 2 | SEQ ID | 36.7 | 1.7 | ok, | ok, |
| | | NO: 6 | | | Fragments | Fragments |
| 7801 | Fn4 + Fn5 variant 1 | SEQ ID | 50.4 | 3.8 | ok, | ok, |
| | | NO: 5 | | | Fragments | Fragments |
| 7803 | Fn4 + Fn5 variant 2 | SEQ ID | 49.7 | 2.2 | ok, | ok |
| | | NO: 7 | | | Fragments | Fragments |

### Example 4

### Protein Purification by Affinity Chromatography and Gel Filtration Chromatography

### Protein A Affinity Chromatography

The expressed and secreted polypeptides were purified by affinity chromatography using the protein A affinity material MabSelectSure (GE Healthcare). Briefly, after centrifugation (10,000 g for 10 minutes) and filtration through a 0.45 μm filter the polypeptide containing clarified culture supernatant was applied on a MabSelectSure column equilibrated with PBS buffer (10 mM Na₂HPO₄, 1 mM KH₂PO₄, 137 mM NaCl and 2.7 mM KCl, pH 7.4). Unbound proteins were removed by washing with equilibration buffer. The polypeptide was eluted with 0.1 M citrate buffer, pH 3.3, and the product containing fractions were neutralized with 1 M TRIS pH 9.0. Afterwards, the solution was dialyzed against 20 mM histidine, 140 mM NaCl, pH 6.0 buffer at 4° C., concentrated with an Amicon Centricon concentration device, and stored in an ice-water bath until further processing.

### Size Exclusion Chromatography

The polypeptide containing solution was applied to a Superdex200 High Load column (GE HealthCare) equilibrated with the same histidine buffer. Fractions were collected. All fractions were analyzed by analytical SEC (Superdex200, GE HealthCare) and fractions with purely monomeric conjugate were pooled and stored frozen at −80° C.

The integrity of the polypeptides were analyzed by SDS-PAGE in the presence and absence of a reducing agent and staining with Coomassie brilliant blue as described in the previous paragraph.

### Example 5

### Binding Assay by Surface Plasmon Resonance

### Instrument: Biacore T100 (GE Healthcare)

### Software: Biacore T100 Control, Version 2.02

Biacore T100 Evaluation, Version 2.02
   Assay format: Chip:CM5-Chip

DCC-fusion protein (Fn5 variant 1; SEQ ID NO: 3) was captured via amine coupled capture molecules. A series with increasing concentrations of netrin-1 was injected.

Chip surface with amine coupled capture molecule alone was used as reference control surface for correction of possible buffer-effects or non specific binding of netrin-1.

Capture molecules: Anti-human IgG antibodies (from goat, Jackson Immuno Research JIR 109-005-098) for Fn5 variant 1.

### Amine Coupling of Capture Molecules

Standard amine coupling according to the manufacturer's instructions: running buffer: HBS-N buffer, activation by mixture of EDC/NHS, aim for ligand density of 5000 RU; the to capture-antibodies were diluted in coupling buffer NaAc, pH 4.5, c=30 μg/mL; finally remaining activated carboxyl groups were blocked by injection of 1 M Ethanolamin.

Kinetic characterization of netrin-1 binding to DCC-fusion protein (Fn5 variant 1; SEQ ID NO: 3) at 25° C.

Running buffer: PBS+0.05% (v/v) Tween 20
Capturing of Fn5 variant 1 on flow cells 2 to 4: Flow 5 μL/min, contact time 72 seconds, c(Fn5 variant 1)=100 nM, diluted with running buffer+1 mg/mL BSA.

### Analyte Sample:

Classical concentration series were measured at a flow rate of 50 μL/min by sequential injection of the analyte in 5 or 6 increasing concentrations between c=400-1 nM. The analyte was injected for 3 minutes followed by a dissociation phase of 20 minutes. Various netrin-1 samples from different manufacturers were used for the measurements (human netrin-1, Alexis 522-100-0000/human metrin-1 Netris Pharma/chicken netrin-1, Alexis 522-106-2010).

Regeneration was performed after each cycle (=each concentration) using 10 mM Glycin pH 1.5, contact time 2 minutes, flow rate 30 μL/min.

FIG. 3 shows, e.g., typical association and dissociation curves of the captured analyte to DCC-fusion protein (Fn5 variant 1; SEQ ID NO: 3) at different concentrations of injected chicken netrin-1.

Kinetic parameters were calculated by using the usual double referencing (control reference: binding of analyte to capture molecule; Flow Cell: netrin-1 concentration “0” as Blank) and calculation with model ‘titration kinetics 1:1 binding.

TABLE 2
Affinity data measured by SPR (BIACORE ® T100) at 25° C.

| | kₐ | k_{d} | t(½) | K_{D} |
|---|---|---|---|---|
| | [M⁻¹s⁻¹] | [s⁻¹] | [min] | [M] |
| Fn5-Fc (IgG1) fusion | 7.9E+04 | 1.3E−04 | 92.1 | 1.6E−09 |
| protein SEQ ID NO: 3 | | | | |
| (Fn5 variant 1)/ | | | | |
| chicken netrin-1 | | | | |
| Fn5-Fc (IgG1) fusion | 3.0E+05 | 5.7E−04 | 20.2 | 1.6E−09 |
| protein SEQ ID NO: 3 | | | | |
| (Fn5 variant 1)/human | | | | |
| netrin-1 | | | | |

### Example 6

### Caspase-3 Activation Assay with H358 Cells

On day 1, cells were plated in serum-free medium (2×10⁵ cells per well in six-well plates with 1 ml medium per well). On day 2, the medium was replaced with 1 ml fresh serum-free medium containing either only vehicle (PBS) or 1 μg/ml mature DCC-fusion protein (Fn5 variant 1, SEQ ID NO: 3) or 1 μg/ml Fn5 variant 1 plus 150 ng/ml netrin-1. Treatments were done on 2 wells per condition. On day 3, the floating as well as all adherent cells from the 2 identically treated wells were harvested as one pool. The cell pellet was resuspended in 55 μL lysis buffer and lysed on ice for 10 min. Then the lysates were pre-cleared by centrifugation at maximum speed for 3 min at 4° C. The supernatants were collected in new tubes and kept on ice during determination of the protein concentration. In a white 96-well plate, the volumes of 30 g protein aliquots were adjusted with lysis buffer to 50 μl final volume. 50 μL reaction mix consisting of 54 μL reaction buffer plus 1 μL DEVD-AFC plus 0.5 μL DTT was added to each well. Fluorescence generation (excitation at 400 nm, emission at 510 nm) was monitored by taking kinetic measurements every 5 min for a total of 1 h. Alternatively, if that was not possible, an initial reading was taken immediately after adding the mix and a final measurement was done after a 1 h incubation at 37° C. (protected from light). All values were normalized to the vehicle control sample. Results are shown in FIG. 4.

### Example 7

### In Vivo Tumor Growth Inhibition of H358 and A549 Xenografts

### H358 Xenografts:

Five-week-old female athymic nu/nu mice were implanted by subcutaneous injection with 5.0×10⁶ H358 cells in 200 μL of PBS into the left flank of the mice to make one tumor per mouse. When tumors reached a volume of approximately 100 mm³, 20 mg/kg of the Fn5 variant 1 fusion protein (SEQ ID NO: 3) (n=11 mice) or an equal volume of buffer (n=12 mice) was injected once weekly intraperitoneally for 4 consecutive weeks. Tumor sizes were measured with a caliper. The tumor volume was calculated with the formula v=0.5 (l×w 2), where v is volume, l is length, and w is width.

### A549 Xenografts:

Five-week-old female athymic nu/nu mice were implanted by subcutaneous injection in the flank with A549 cells. When tumors reached a volume of approximately 100 mm³ the Fn5 variant 1 fusion protein (SEQ ID NO: 3) at a dose of 20 mg/kg or an equal volume of buffer was injected either once or twice per week intraperitoneally for 4 consecutive weeks (n=10 mice per treatment group). Tumor sizes were measured with a caliper. The tumor volume was calculated with the formula v=0.5 (l×w 2), where v is volume, l is length, and w is width. See also FIG. 5 (“Trap” means variant 1 fusion protein (SEQ ID NO: 3)).

1-11. (canceled)

12. A DCC-fusion protein comprising the amino acid sequence of SEQ ID NO: 2

13. A DCC-fusion protein comprising the amino acid sequence of SEQ ID NO: 3.

14. A nucleic acid molecule encoding the DCC-fusion protein according to claim 12.

15. A nucleic acid molecule encoding the DCC-fusion protein according to claim 13.

16. The nucleic acid molecule according to claim 12 which comprises the nucleotide sequence of SEQ ID NO: 1.

17. The nucleic acid molecule according to claim 13 which comprises the (new) nucleotide sequence of SEQ ID NO: 1.

18. A vector containing the nucleic acid according claim 14 capable of expressing said nucleic acid in a eukaryotic host cell.

19. A vector containing the nucleic acid according claim 15 capable of expressing said nucleic acid in a eukaryotic host cell.

20. A host cell comprising the nucleic acid molecule according to claim 14.

21. A host cell comprising the nucleic acid molecule according to claim 15.

22. A host cell comprising the nucleic acid molecule according to claim 18.

23. A host cell comprising the nucleic acid molecule according to claim 19.

24. A method for producing a DCC-fusion protein said method comprising:
expressing a nucleic acid in a eukaryotic host cell; and recovering the DCC-fusion protein from said cell or the cell culture supernatant.

25. A method according to claim 24, wherein said DCC-fusion protein has the amino acid sequence of SEQ ID NO: 2.

26. The method according to claim 24, wherein said DCC-fusion protein has the amino acid sequence of SEQ ID NO: 3.

27. A pharmaceutical composition comprising: a DCC-fusion protein, a vector or a host; and a pharmaceutically acceptable carrier.

28. The pharmaceutical composition of claim 16, wherein said DCC-fusion protein has the amino acid sequence of SEQ ID NO: 2; and wherein said vector has a nucleic acid encoding the DCC-fusion protein of SEQ ID NO: 2.

29. The pharmaceutical composition of claim 16, wherein said DCC-fusion protein has the amino acid sequence of SEQ ID NO: 3; and wherein said vector has a nucleic acid encoding the DCC-fusion protein of SEQ ID NO: 3.

30. A method of treating cancer in a subject, said method comprising:
administering to said subject a DCC-fusion protein selected from the group consisting of a DCC-fusion protein having a sequence of SEQ ID NO: 2, SEQ ID NO: 3 or combinations thereof.

31. The method of claim 19, wherein said cancer is selected from the group consisting of colorectal cancer, non-small cell lung cancer, or metastatic breast cancer.


SEQUENCE LISTING (7 sequences)

SEQ ID NO: 1 - DNA, 1084 bp, Artificial Sequence
  Source: /Note=“Description of Artificial Sequence DNA encoding the DCC-fusion protein (Fn5-Fc fusion protein) 7800”
aagcttgccgccaccatgggatggagctgtatcatcctcttcttggtagcaacagctacaggtgtccactccagcacccccatgctgcctccagtgggcgtccaggccgtggctctcacacacgacgcagtccgcgtgtcctgggccgataactctgttcccaagaatcagaaaacctcagaagtgagactgtacactgtccgctggcggacatccttctccgcttctgcaaagtataagagtgaagacaccactagcctttcctacaccgccacagggctgaaacctaacaccatgtatgagttttctgtgatggtaacaaagaataggagatcaagcacctggtccatgactgctcatgcaacaacctacgaggccgctccaaaatcttgtgacaaaactcacacatgtccaccgtgcccagcacctgaactcctggggggaccgtcagtcttcctcttccccccaaaacccaaggacaccctcatgatctcccggacccctgaggtcacgtgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggcaaatgagctagcg

SEQ ID NO: 2 - protein, 353 aa, Artificial Sequence
  Source: /Note=“Description of Artificial Sequence Amino acid sequence of the DCC-fusion protein (Fn5-Fc fusion protein) 7800”
MGWSCIILFLVATATGVHSSTPMLPPVGVQAVALTHDAVRVSWADNSVPKNQKTSEVRLYTVRWRTSFSASAKYKSEDTTSLSYTATGLKPNTMYEFSVMVTKNRRSSTWSMTAHATTYEAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 334 aa, Artificial Sequence
  Source: /Note=“Description of Artificial Sequence Amino acid sequence of mature DCC-fusion protein (Fn5-Fc fusion protein) 7800”
STPMLPPVGVQAVALTHDAVRVSWADNSVPKNQKTSEVRLYTVRWRTSFSASAKYKSEDTTSLSYTATGLKPNTMYEFSVMVTKNRRSSTWSMTAHATTYEAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 334 aa, Artificial Sequence
  Source: /Note=“Description of Artificial Sequence Amino acid sequence of mature Fn5-Fc fusion protein 7809”
STPMLPPVGVQAVALTHDAVRVSWADNSVPKNQKTSEVRLYTVRWRTSFSASAKYKSEDTTSLSYTATGLKPNTMYEFSVMVTKNRRSSTWSMTAHATTYEAAPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 451 aa, Artificial Sequence
  Source: /Note=“Description of Artificial Sequence Amino acid sequence of mature Fn4-Fn5-Fc fusion protein 7801”
QVPDQPSSLHVRPQTNCIIMSWTPPLNPNIVVRGYIIGYGVGSPYAETVRVDSKQRYYSIERLESSSHYVISLKAFNNAGEGVPLYESATTRSITDPTDPVDYYPLLDDFPTSVPDLSTPMLPPVGVQAVALTHDAVRVSWADNSVPKNQKTSEVRLYTVRWRTSFSASAKYKSEDTTSLSYTATGLKPNTMYEFSVMVTKNRRSSTWSMTAHATTYEAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 333 aa, Artificial Sequence
  Source: /Note=“Description of Artificial Sequence Amino acid sequence of mature Fn5-Fc fusion protein 7802”
STPMLPPVGVQAVALTHDAVRVSWADNSVPKNQKTSEVRLYTVRWRTSFSASAKYKSEDTTSLSYTATGLKPNTMYEFSVMVTKNRRSSTWSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 450 aa, Artificial Sequence
  Source: /Note=“Description of Artificial Sequence Amino acid sequence of mature Fn4-Fn5-Fc fusion protein 7803”
QVPDQPSSLHVRPQTNCIIMSWTPPLNPNIVVRGYIIGYGVGSPYAETVRVDSKQRYYSIERLESSSHYVISLKAFNNAGEGVPLYESATTRSITDPTDPVDYYPLLDDFPTSVPDLSTPMLPPVGVQAVALTHDAVRVSWADNSVPKNQKTSEVRLYTVRWRTSFSASAKYKSEDTTSLSYTATGLKPNTMYEFSVMVTKNRRSSTWSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK